(19) United States
(12) Patent Application Publication
(10) Pub. No.: US 2018/0321233 A1
(43) Pub. Date: Nov. 8, 2018

(54) System, Device and Method for Counting Desired Cells in a Body Fluid

(71) Applicant: Sanguis LLC, Philadelphia, PA (US)

(72) Inventors: Daniel Zhang, Philadelphia, PA (US); Divyansh Agarwal, Philadelphia, PA (US); Prateek Agarwal, Philadelphia, PA (US)

(21) Appl. No.: 15/968,120

(22) Filed: May 1, 2018

Related U.S. Application Data
(60) Provisional application No. 62/500,432, filed on May 2, 2017.

(51) Int. Cl. G01N 33/543; G01N 33/58; B01L 3/00; G01N 15/14

(52) U.S. Cl. CPC G01N 33/54386; G01N 33/582; B01L 3/502715; G01N 15/1404; G01N 15/1484; B01L 2300/0816; G01N 2015/1486; B01L 2400/0487; B01L 2300/027; B01L 2200/0647; B01L 2200/16; B01L 2300/16; B01L 2400/06

(57) ABSTRACT

A system, a microfluidic chip and a method are provided for counting desired cells in a body fluid that allows for a reasonable range of error in exchange for fast and cheap diagnosis. The fluid containing the cells to be measured is immobilized on a microfluidic chip and stained and the cell count is determined from optical signals that measure the amount of stain acquired by the immobilized cells.

### RELATED APPLICATION

This application claims the benefit of and priority to U.S. Provisional Application Ser. No. 62/500,432, filed May 2, 2017, the disclosure of which is hereby incorporated herein by reference in its entirety.

### FIELD

The present invention relates to the field of human diagnostics, in general, and to fluorescence-based analysis of cells, in particular.

### BACKGROUND

There is a great need for fast, inexpensive, and widely accessible medical diagnostics, particularly in the area of home health monitoring technologies. Significant morbidity and mortality are incurred when medical conditions are identified late and patients are not able to receive timely treatment. Furthermore, delayed diagnoses of certain medical conditions are also associated with tremendous amount of excess healthcare costs because these patients often require emergent treatment or critical care. For example, medical complications due to a failure in identifying low blood cell counts in patients receiving chemotherapy is associated with an estimated 10,000 deaths and $600 million in medical expenses in 2017 alone in the USA.

One way of screening for medical conditions is to measure certain cell counts. For example, for the estimated 650,000 patients who receive outpatient chemotherapy each year in the USA, low blood cell counts are a common and serious side effect of their treatment. In particular, low levels of neutrophils, a type of white blood cell that defends against bacterial and fungal organisms, is closely associated with developing serious and sometimes atypical infections. Therefore, methods for rapid and inexpensive monitoring of a patient's cell counts would be highly desirable because it would enable patients to identify low counts at the earliest stage possible and offer a unique opportunity for medical intervention to prevent serious medical complications.

These prior art methods allow for high precision measurements. However, as a trade off, speed and cost efficiency are sacrificed.

The enzyme linked immunosorbent assay (ELISA) is a technique designed for detecting molecules such as small molecules, vitamins, peptides, and proteins. U.S. Pat. No. 9,546,211B2, to Singh, describes a method of measuring the levels of an anti-TNF-alpha sdAb in a sample from a subject using the ELISA. European Patent Application 3103812A1, to Gutierrez et al., describes antibody-sandwich ELISA methods and kits for vascular endothelial growth factor (VEGF) as an antigen to detect types of VEGF levels in biological samples from animal models and human patients, which can be used as a diagnostic or prognostic index. ELISA is a relatively inexpensive, rapid, and reliable method that may be adapted to counting cells. However, Applicants are not aware that anyone has modified the ELISA methods for counting cells.

### SUMMARY

A system is provided for counting desired cells in a body fluid. In some embodiments, the system includes a microfluidic chip having a capture chamber for immobilizing the desired cells and receiving a labeling solution wherein the labeling solution interacts with an immobilized substance in the capture chamber and produces an optical signal. An optoelectronic unit can detect the optical signal from the capture chamber that corresponds to the photochemical properties of the labeling molecule, and a microprocessor can control the microfluidic chip and the optoelectronic unit, and can process the optical signal measured by the optoelectronic unit to calculate a cell count.

In some embodiments, the microfluidic chip can include a first reservoir that collects the body fluid, a section coated with an anti-coagulating agent, a first mechanism that controls the volume of the body fluid, the capture chamber, a second mechanism that allows the body fluid to flow unidirectionally from the first mechanism to the capture chamber, a first container of the labeling solution, which releases the labeling solution into the capture chamber, and a second reservoir that collects waste.

In some embodiments, the immobilized substance in the capture chamber comprises the desired cells, wherein the labeling solution interacts with the desired cells and produces the optical signal. In some embodiments, immobilized substance in the capture chamber comprises enzymes of the antibody-enzyme solution, and wherein the labeling solution reacts with the enzymes in the antibody-enzyme solution. In some embodiments, the anti-coagulating agent is Ethylenediaminetetraacetic acid (EDTA). In some embodiments, the anti-coagulating agent is citrate, heparin, Vitamin E, or hementin.

In some embodiments, the system also includes a second container of a first wash solution that can release the first wash solution into the capture chamber to wash away excess body fluid. In some embodiments, the microfluidic portion of the system does not contain a first wash solution. In some embodiments, the second container can release the first wash solution into the capture chamber to wash away excess antibody-enzyme solution.

In some embodiments, the first mechanism comprises a reservoir with a gutter. In some embodiments, the first mechanism comprises a capillary glass tube with preset volume to deliver blood into an opening channel on the microfluidic chip. In some embodiments, the first mechanism comprises a pressure/volume sensing valve that closes once a set amount of blood is delivered into a reservoir. In some embodiments, the second mechanism comprises a one-way flow valve. In some embodiments the second mechanism comprises a programmable microfluidic pump. In some embodiments the programmable microfluidic pump is a peristaltic microfluidic pump.

In some embodiments, the microfluidic chip also includes a third container of an antibody-enzyme solution that can release the antibody-enzyme solution into the capture chamber to react with the desired cells in the capture chamber. In some embodiments, the microfluidic chip also includes a fourth container of a second wash solution that can release the second wash solution into the capture chamber to wash away excess antibody-enzyme solution. In some embodiments, the microfluidic portion of the system does not contain the second wash solution.

In some embodiments, the capture chamber comprises a solid scaffold, and recognition molecules that selectively bind to the desired cells. In some embodiments, the solid scaffold is treated with ultraviolet radiation and activating reagents, and the treated solid scaffold binds with the recognition molecules. In some embodiments, the substance in the capture chamber further comprises a linker. The linker can include one chemical moiety or a linear arrangement of a plurality of chemical moieties, such that the linker has strong binding affinity to the solid scaffold on one end and the recognition molecules on the other end.

In some embodiments, the desired cells are neutrophils. In some embodiments, the desired cells are any cell that has a unique surface molecular marker that can be targeted, such as CD4 T-cells, CD8 T-cells, B-cells, or natural killer cells.

In some embodiments, the first container is in the form of a first syringe. In some embodiments, the first container comprises a container connected to a pump. In some embodiments, the first container comprises a container with a flow valve whereby fluid moves by capillary action after opening of the flow valve. In some embodiments, the second container is in the form of a second syringe. In some embodiments, the second container comprises a container connected to a pump. In some embodiments, the second container comprises a container with a flow valve whereby fluid moves by capillary action after opening of the flow valve. In some embodiments, the third container is in the form of a third syringe. In some embodiments, the third container comprises a container connected to a pump. In some embodiments, the third container comprises a container with a flow valve whereby fluid moves by capillary action after opening of the flow valve. In some embodiments, the fourth container is in the form of a fourth syringe. In some embodiments, the fourth container comprises a container connected to a pump. In some embodiments, the fourth container comprises a container with a flow valve whereby fluid moves by capillary action after opening of the flow valve.

In some embodiments, the optoelectronic unit comprises a light source and an optical detection unit. The light source can emit light that that corresponds to the photochemical properties of the labeling molecule. The optical detection unit can detect the optical signal from the capture chamber that corresponds to the photochemical properties of the labeling molecule.

In some embodiments, the system can also include a display and a memory that stores instructions. The microprocessor can process the instructions to detect the presence of the microfluidic chip, detect entry of the body fluid into the capture chamber, engage the first container after the desired cells are immobilized in the capture chamber to release the labeling solution; receive the optical signal from the optical detection unit, compare the optical signal to a pre-programmed algorithm to calculate the cell count, and display the cell count on the display.

In some embodiments, the system can also include a display and a memory that stores instructions, and the microprocessor can process the instructions to engage the second container to release the first wash solution after the desired cells are immobilized in the capture chamber and before the first container releases the labeling solution. In some embodiments, the system can also include a display and a memory that stores instructions, and the microprocessor can process the instructions to engage the third container to release the antibody-enzyme solution after the desired cells are immobilized in the capture chamber and before the first container releases the labeling solution. In some embodiments, the system can also include a display and a memory that stores instructions, and the microprocessor can process the instructions to engage the fourth container to release the second wash after the antibody-enzyme solution enters into the capture chamber and before the first container releases the labeling solution.

A method is also provided that modifies the enzyme linked immunosorbent assay (ELISA) method to count desired cells in a body fluid. In some embodiments, the method can include immobilizing the desired cells in a capture chamber on a microfluidic chip (step a), and adding a labeling solution to the capture chamber (step b). The labeling solution can interact with immobilized substance in the capture chamber and produces an optical signal. The method also includes detecting the optical signal from the capture chamber that correspond to the photochemical properties of the labeling molecule (step c), and providing a cell count based on the optical signal (step d).

In some embodiments, the method also includes, before conducting steps (a) to (c), treating a solid scaffold of the capture chamber with ultraviolet radiation and activating reagents, and attaching recognition molecules that bind to the treated solid scaffold. In some embodiments, the method also includes, before conducting steps (a) to (c), attaching to the solid scaffold of the capture chamber a linker, and attaching recognition molecules to the linker. The linker can include one chemical moiety or a linear arrangement of a plurality of chemical moieties. The linker can have strong binding affinity to the solid scaffold on one end and the recognition molecules on the other end. In some embodiments, the method also includes, before conducting steps (a) to (c), adding the body fluid to the microfluidic chip, mixing the body fluid with an anti-coagulating agent, and controlling the volume of the body fluid before the body fluid enters into the capture chamber.

In some embodiments, the anti-coagulating agent is Ethylenediaminetetraacetic acid (EDTA). In some embodiments, the anti-coagulating agent is citrate, heparin, Vitamin E, or hementin. In some embodiments, the desired cells are neutrophils. In some embodiments, the desired cells are any cell that has a unique surface molecular marker that can be targeted, such as CD4 T-cells, CD8 T-cells, B-cells, or natural killer cells.

In some embodiments, step (a) comprises reacting recognition molecules with the desired cells in the body fluid. In some embodiments, the method also includes, after step (a) and before step (b), washing away excess body fluid after reacting the recognition molecules with the desired cells in the body fluid. In some embodiments, the method does not contain a first wash solution.

In some embodiments, step (b) comprises adding the labeling solution to the capture chamber, and reacting the labeling solution with the immobilized substance in the capture chamber. The immobilized substance in the capture chamber can comprise desired cells, wherein the labeling solution interacts with the desired cells and produces the optical signal. In some embodiments, step (b) comprises adding an antibody-enzyme solution to the capture chamber; adding the labeling solution to the capture chamber, and reacting the labeling solution with the immobilized substance in the capture chamber. The immobilized substance in the capture chamber can comprise enzymes in the antibody-enzyme solution. In some embodiments, step (b) comprises adding an antibody, followed by a secondary antibody-enzyme conjugate, followed by a labeling solution.

In some embodiments, the method further comprises adding a second wash solution to wash away excess antibody-enzyme solution, after adding the antibody-enzyme solution and before adding the labeling solution to the capture chamber. In some embodiments, the method does not contain the second wash solution.

In some embodiments, step (c) comprises emitting light that correspond to the photochemical properties of the labeling molecule, and detecting the optical signal from the capture chamber constituting light that that correspond to the photochemical properties of the labeling molecule. In some embodiments, step (d) comprises comparing the optical signal to a pre-determined algorithm to calculate the cell count, and displaying the cell count.

In some embodiments, a microfluidic chip comprises a capture chamber in which desired cells are immobilized, a first container of labeling solution that is released into the capture chamber wherein the labeling solution interacts with substance in the capture chamber and then becomes fluorescent; a first reservoir that collects the body fluid, a first mechanism that controls the volume of the body fluid, and a second mechanism that allows the body fluid to flow unidirectionally from the first mechanism to the capture chamber.

In some embodiments, the microfluidic chip further comprises a section coated with an anti-coagulating agent and a second reservoir that collects waste. In some embodiments, the immobilized substance in the capture chamber comprises the desired cells, wherein the labeling solution interacts with the desired cells and produces the optical signal. In some embodiments, the microfluidic chip further comprises a second container of a first wash solution, which releases the first wash solution into the capture chamber to wash away excess body fluid. In some embodiments, the microfluidic portion of the system does not contain a first wash solution. In some embodiments, the anti-coagulating agent is Ethylenediaminetetraacetic acid (EDTA). In some embodiments, the anti-coagulating agent is citrate, heparin, Vitamin E, or hementin.

In some embodiments, the first mechanism comprises a reservoir with a gutter. In some embodiments, the first mechanism comprises a capillary glass tube with preset volume to deliver blood into an opening channel on the microfluidic chip. In some embodiments, the first mechanism comprises a pressure/volume sensing valve that closes once a set amount of blood is delivered into a reservoir. In some embodiments, the second mechanism comprises a one-way flow valve. In some embodiments the second mechanism comprises a programmable microfluidic pump. In some embodiments the programmable microfluidic pump is a peristaltic microfluidic pump. In some embodiments, the microfluidic chip further comprises a third container of an antibody-enzyme solution, which releases the antibody-enzyme solution into the capture chamber to react with the desired cells in the capture chamber. In some embodiments, the immobilized substance in the capture chamber comprises enzymes of the antibody-enzyme solution, and wherein the labeling solution reacts with the enzymes in the antibody-enzyme solution. In some embodiments, the microfluidic chip further comprises a fourth container of a second wash solution, which releases the second wash solution into the capture chamber to wash away excess antibody-enzyme solution. In some embodiments, the microfluidic chip does not contain the second wash solution.

In some embodiments, the capture chamber comprises a solid scaffold and recognition molecules that selectively bind to the desired cells. In some embodiments, the solid scaffold is treated with ultraviolet radiation and the treated solid scaffold binds with the recognition molecules. In some embodiments the immobilized substance in the capture chamber further comprises a linker, the linker comprising one chemical moiety or a linear arrangement of a plurality of chemical moieties, wherein the linker has strong binding affinity to the solid scaffold on one end and the recognition molecules on the other end. In some embodiments, the desired cells are neutrophils. In some embodiments, the desired cells are any cell that has a unique surface molecular marker that can be targeted, such as CD4 T-cells, CD8 T-cells, B-cells, or natural killer cells. In some embodiments, the second container releases the first wash solution into the capture chamber to wash away excess antibody-enzyme solution

In some embodiments, the first container comprises a first syringe. In some embodiments, the second container comprises a second syringe. In some embodiments, the third container comprises a third syringe. In some embodiments, the fourth container comprises a fourth syringe

In some embodiments, a capture chamber for use in counting desired cells in a body fluid comprises a solid scaffold, and recognition molecules that selectively bind to the desired cells. In some embodiments, the solid scaffold is treated with ultraviolet radiation and activating reagents, and the treated solid scaffold binds with the recognition molecules. In some embodiments, the substance in the capture chamber also includes a linker. The linker can comprise one chemical moiety or a linear arrangement of a plurality of chemical moieties, and the linker can have strong binding affinity to the solid scaffold on one end and the recognition molecules on the other end. In some embodiments the desired cells are neutrophils. In some embodiments, the desired cells are any cell that has a unique surface molecular marker that can be targeted, such as CD4 T-cells, CD8 T-cells, B-cells, or natural killer cells. In some embodiments, the capture chamber also includes a labeling solution that interacts with the desired cells and produces an optical signal. In some embodiments, the capture chamber also includes an antibody-enzyme solution that interacts with the desired cells. In some embodiments, the labeling solution interacts with the antibody-enzyme solution.

### BRIEF DESCRIPTION OF DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates methods for connecting a solid scaffold via a recognition molecule to a desired cell in accordance with some embodiments.

FIGS. 2A, 2B, and 2C illustrate methods for staining the desired cell in accordance with some embodiments.

FIG. 3 illustrates the design of the microfluidic chip in accordance with some embodiments.

FIG. 4 illustrates an electronic reader with the microfluidic chip placed within for measurements in accordance with some embodiments.

FIG. 5A is a simplified flowchart of a method for counting desired cells in a body fluid where the endogenous staining protocol is desired in accordance with some embodiments.

FIG. 5B is a simplified flowchart of a method for counting desired cells in a body fluid where the exogenous “direct pathway” protocol is desired in accordance with some embodiments.

FIG. 5C is a simplified flowchart of a method for counting desired cells in a body fluid where the exogenous “indirect pathway” protocol is desired in accordance with some embodiments.

FIG. 6A is a simplified flowchart describing the workflow of the microprocessor where the endogenous staining protocol is desired in accordance with some embodiments.

FIG. 6B is a simplified flowchart describing the workflow of the microprocessor where the exogenous “direct pathway” protocol is desired in accordance with some embodiments.

FIG. 6C is a simplified flowchart describing the workflow of the microprocessor where the exogenous “indirect pathway” protocol is desired in accordance with some embodiments.

FIG. 7 is a diagram showing an exemplary computer system suitable for use with the methods and systems of the present disclosure.

In all the figures similar reference numerals identify similar parts.

### DETAILED DESCRIPTION

A system and method for counting desired cells in a body fluid is provided. In some embodiments, the system includes a microfluidic chip on which the desired cells are immobilized and stained. A measured optical signal relating to the immobilized and stained desired cells is then used to calculate the cell count. In some embodiments, a labeling molecule interacts with the desired cells and produces a measurable optical signal. In some embodiments, the labeling molecule interacts with exogenously supplied enzymes that attach to the desired cells to generate the optical signal.

In some embodiments, the microfluidic chip comprises a capture chamber. In some embodiment, the capture chamber inIn some embodimentscludes a solid scaffold and recognition molecules that selectively bind to the desired cells. In some embodiments, the recognition molecules are directly attached to the solid scaffold after activation of the solid scaffold surface with ultraviolet radiation and activating reagents. In some embodiments, the recognition molecules are linked to the solid scaffold by a linker, i.e., one chemical moiety or a linear arrangement of a plurality of chemical moieties that has a sufficiently strong binding affinity to the solid scaffold on one end and to the recognition molecules on the other end.

The system can also include a mechanism for counting desired cells in a body fluid. The system comprises the microfluidic chip with the capture chamber, an optoelectronic unit that detects optical signals from the microfluidic chip, as well as a microprocessor that controls the microfluidic chip and the optoelectronic unit and that calculates the cell count based on the measured optical signals. In some embodiments, the optoelectronic unit comprises a light source and an optical detection unit. The light source emits light that corresponds to the appropriate photochemical properties of the labeling molecule, and the optical detection unit detects the optical signal from the capture chamber that correspond to the appropriate photochemical properties of the labeling molecule. In some embodiments, the actions of the microprocessor comprise detecting the presence of the microfluidic chip and the entry of the body fluid into the capture chamber, engaging components on the microfluidic chip, receiving the optical signal from the optical detection unit, determining a cell count based on the optical signal, and displaying the cell count on a display.

A method is also provided for modifying the enzyme linked immunosorbent assay (ELISA) method to determine the quantity of the desired cells in a body fluid. The method comprises the steps of immobilizing the desired cells in the capture chamber on the microfluidic chip, treating with immobilized cells with a labeling molecule, the labeling molecule interacting with the immobilized substances, measuring the optical signal from the capture chamber that are associated with the labeling molecule, and calculating a cell count based on the optical signal.

In some embodiments, the capture chamber is made by treating a solid scaffold of the capture chamber with ultraviolet radiation and chemical reagents to further activate the surface, and then attaching recognition molecules that bind to the treated solid scaffold. In some embodiments, the capture chamber is manufactured by attaching to the solid scaffold a linker, i.e., one chemical moiety or a linear arrangement of a plurality of chemical moieties that has strong binding affinity to the solid scaffold on one end and the recognition molecules on the other end, and then attaching recognition molecules to the linker.

In some embodiments, the method comprises a novel application of the conventional ELISA to cells. The method further comprises a novel modification to the conventional ELISA where the labeling molecule interacts directly with the desired cells and produces a measurable optical signal. In some embodiments, the method further comprises generating light that corresponds to the appropriate photochemical property of the labeling molecule and then detecting the optical signal from the capture chamber that correspond to the appropriate photochemical properties of the labeling molecule. The method may further comprise applying the measured optical signal to a pre-determined algorithm to calculate the cell count and then displaying the cell count.

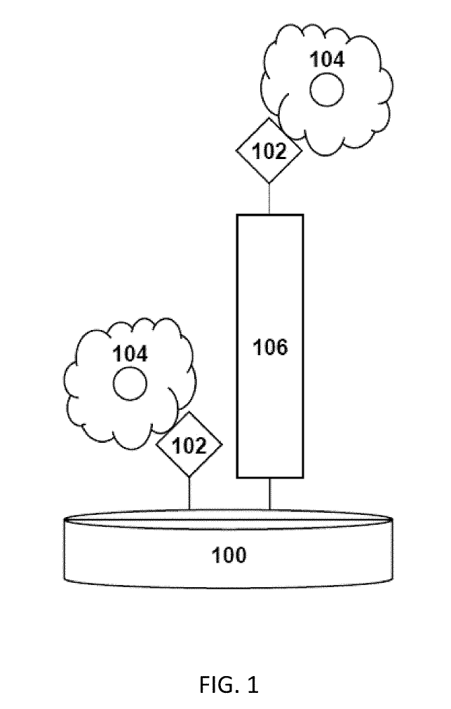

FIG. 1 illustrates methods for connecting a solid scaffold 100 via a recognition molecule 102 to a desired cell 104. The solid scaffold 100 may be composed of glass or polymer, such as polydimethylsiloxane (PDMS) or poly(methyl methacrylate) (PMMA), or other materials including thermoplastics such as polycarbonate. The recognition molecule 102 may be antibodies, proteins, small molecules, or peptides that selectively bind to a desired cell type and/or to a linker 106. One end of the recognition molecule 102 binds sufficiently tightly, sufficiently selectively, and sufficiently specifically to a desired cell 104. The other end of the recognition molecule 102 is attached to the solid scaffold 100 either directly or via linker 106.

In some embodiments, the linker 106 comprises one chemical moiety or an arrangement of a plurality of other chemical moieties. In some embodiments, the linker 106 can provide additional distance between the solid support and the recognition molecule. These increased degrees of freedom and increased flexibility can improve the efficiency of this process. In some embodiments, the linker 106 can be a linear molecule, water soluble, or both. In some embodiments, the linker 106 can have orthogonal reactive handles on either end so as to allow for facile and selective chemical functionalization. In some embodiments, the linker 106 can comprise a bifunctionalized polyethyleneglycol (PEG) derivatives. In some embodiments, the linker 106 comprises three moieties: 3-mercaptopropyl trimethoxysilane, N-y-maleimidobutyryloxy succinimide ester (GMBS), and NeutrAvidin.

In some embodiments, the recognition molecule 102 is directly attached to the solid scaffold 100. The surface of the solid scaffold 100 is activated such that the recognition material 102 can bind directly and sufficiently tightly to the solid scaffold 100. In some embodiments, the solid scaffold 100 comprises PMMA. In some embodiments, the recognition molecule 102 is an antibody or protein. In some embodiments, the surface of the solid scaffold 100 is activated by irradiation with UV light (254 nm with 15 mW cm⁻² fluence for 10 min) The surface of the solid scaffold 100 is then further activated by treating with an activating solution of 1-ethyl-3-[3-dimethylaminopropyl] carbodiimide hydrochloride and N-hydroxysuccinimide. Then the activating solution is removed and a solution of the recognition molecule 102 is applied to the solid scaffold 100. Afterwards the solution of the recognition molecule 102 is removed, and the solid scaffold 100 is cleaned. For example, the surface of the solid scaffold 100 is activated by irradiation with UV light (254 nm with 15 mW cm⁻² fluence for 10 min) The surface of the solid scaffold 100 is then further activated by treating with an activating solution of 25 mM 1-ethyl-3-[3-dimethylaminopropyl] carbodiimide hydrochloride and 50 mM N-hydroxysuccinimide in 150 mM 2-(4-morpholino)-ethane sulfonic acid at pH 6 for 1 hour. Then the activating solution is removed and a solution of the recognition molecule 102 (1-1000 μg/mL) is applied to the solid scaffold 100 in 150 mM phosphate buffered saline (PBS) at pH 7.4 for 4 hours. Afterwards the solution of the recognition molecule 102 is removed, and the solid scaffold 100 is cleaned with PBS. In some embodiments, this method is based on that described by Adams, et al. (Adams, et al. J. Am Chem Soc. 2008).

In some embodiments, the recognition molecule 102 is indirectly attached to the solid scaffold 100 by using the linker 106. The linker 106 has strong binding affinity to both the solid scaffold 100 and the recognition molecule 102. In some embodiments, the linker 106 is attached to both the solid scaffold 100 and the recognition molecule 102. In some embodiments, the solid scaffold 100 comprises PDMS and the recognition molecule 102 is an antibody or protein. In some embodiments, the solid scaffold 100 is first treated with 3-mercaptopropyl trimethoxysilane. Excess 3-mercaptopropyl trimethoxysilane is removed by flushing the solid scaffold 100 with solvent, such as ethanol. The solid scaffold 100 is then treated with GMBS. The solid scaffold 100 is then treated with NeutrAvidin, Avidin, or strepavidin. For example, the solid scaffold 100 is treated with a 0.25 mg/mL solution of GMBS in dimethylsulfoxide (DMSO) at room temperature for 15 min. Then the solution of GMBS is removed by flushing 100 with ethanol. The solid scaffold 100 is then treated with a solution of 0.1% (v/v) NeutrAvidin, Avidin, or strepavidin (reconstituted from lyophilized powder as per the manufacturer's instructions) in PBS for 18 h at 4° C. The solution of NeutrAvidin is removed by flushing the solid scaffold 100 with PBS. The solid scaffold 100 is then flushed with a solution of bovine serum albumin (BSA) or HSA in PBS. The solid scaffold 100 is then treated with a solution of 102. For example, the solid scaffold 100 is flushed with a solution of 0.1% bovine serum albumin (BSA) in PBS, and the solid scaffold 100 is then treated with a 0.1-100 μg/mL solution of 102 in PBS at room temperature for 2 hours. Finally, the solution of the recognition molecule 102 is removed by flushing the solid scaffold 100 with PBS. In some embodiments, this method is based on that described by Murthy, et al (Murthy, et al. Langmuir 2004).

In some embodiments, once the recognition molecule 102 is attached to the solid scaffold 100, the solid scaffold 100 can be stored under moist conditions at or below 25° C. In some embodiments, the desired cell 104 is isolated by treating the solid scaffold 100 with a test sample, which may include body fluids such as blood, cerebrospinal fluid (CSF), pleural fluid, ascites fluid, or urine. In some embodiments, the desired cell 104 attaches to the recognition molecule 102 and is immobilized, whereas undesired components within the body fluids remain in solution or suspended. The undesired components are removed by flushing the solid scaffold 100 with PBS, leaving behind the desired cell 104 for further analysis.

In some embodiments, the desired cell 104 is the neutrophil. Other desired cells may include CD4⁺ T-cells, circulating tumor cells, CAR T-cells, or eosinophils. Many other cell types can also be detected and counted using the systems and methods of the present disclosure.

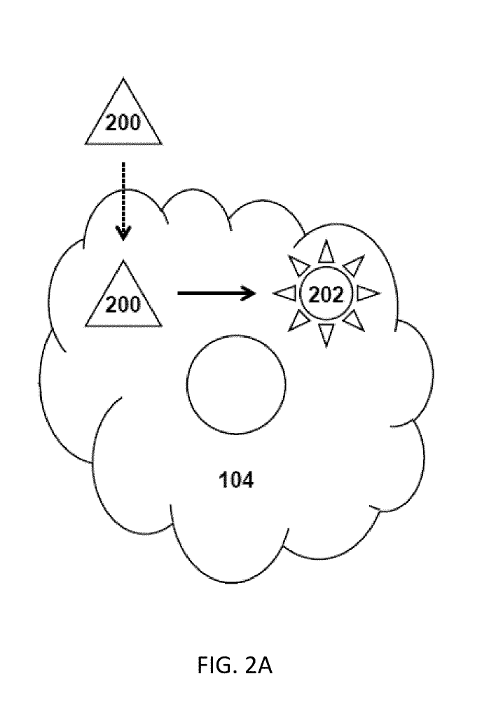

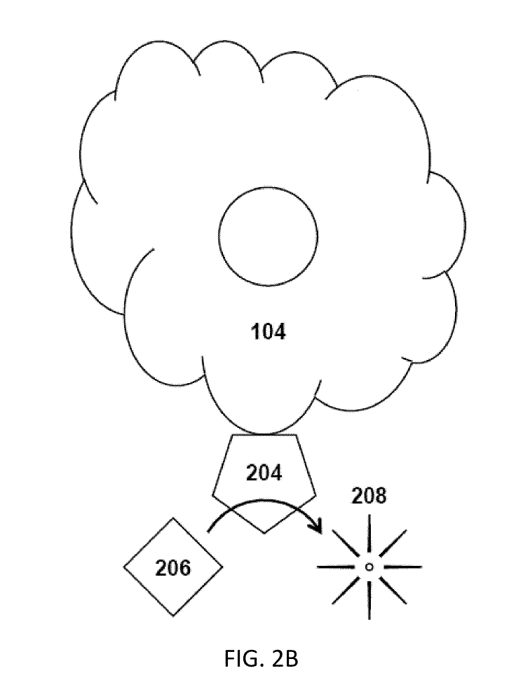

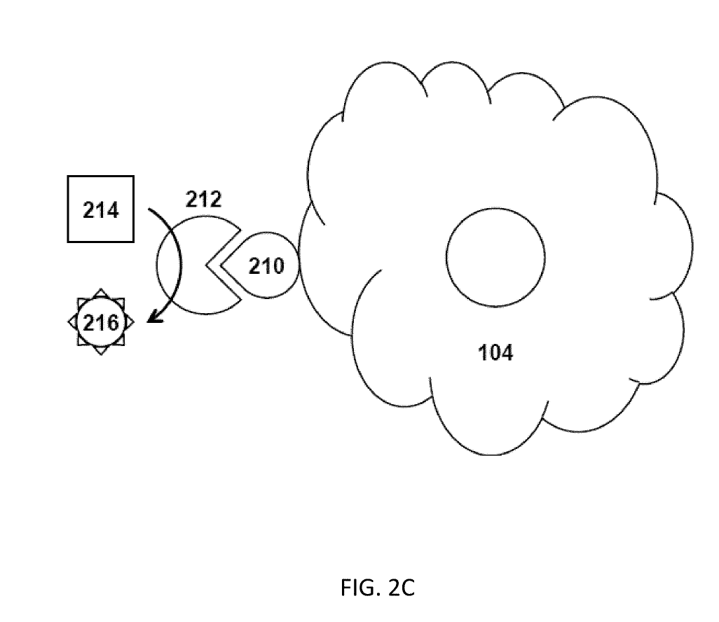

FIGS. 2A, 2B, and 2C illustrate methods for staining the desired cell 104. In some embodiments. The staining is enabled by optically active molecules or molecules that undergo a chemical transformation that changes their photochemical properties. In some embodiments, the photochemical properties comprise excitation and emission spectra of the optically active molecules or the molecules that undergo the chemical transformation if the optically active molecules or the molecules that undergo the chemical transformation are fluorescent. Alternatively, the photochemical properties comprise colorimetric properties of the optically active molecules or the molecules that undergo the chemical transformation.

In some embodiments, the labeling molecule can be fluorogenic, that is, they are non-fluorescent or weakly fluorescent until chemically transformed into strongly fluorescent molecules, such as by some enzyme action or other reaction. In some embodiments, the labeling molecule may be a cell viability stain or label.

In FIG. 2A, the transformation is achieved by an endogenous staining protocol. In some embodiments, labeling molecules can be cell-permeable and non- or weakly-fluorescent. Upon entering the cell, such labeling molecules can be chemically transformed into strongly fluorescent molecules. In some embodiments, a first labeling molecule 200 is transformed into a first stain molecule 202. In some embodiments, the transformation from the first labeling molecule 200 to the first stain molecule 202 is mediated by particular enzymes naturally occurring within the desired cell 104. In some embodiments, the first labeling molecule 200 is non-fluorescent or weakly fluorescent, whereas the first stain molecule 202 is moderately or brightly fluorescent. The first labeling molecule 200 is non-toxic. The first labeling molecule 200 preferably does not interfere with important cellular pathways and processes and is sufficiently cell-permeable. The first stain molecule 202 is also non-toxic. The first stain molecule 202 also preferably does not interfere with important cellular pathways and processes and is sufficiently cell-impermeable. In some embodiments, the first labeling molecule 200 may be fluorescein diacetate (FDA), which can be transformed by endogenous esterases into the first stain molecule 202, which may be fluorescein. The first labeling molecule 200 can also be in the form of related derivatives of FDA, including dichlorofluorescein diacetate and diacetoxymethyl fluorescein. The desired cell 104 is treated with FDA, which may not need to be removed for further analysis. In some embodiments, one or more of Hoechst 33342, difluorofluorescein diacetate, diacetoxymethyl dichlorofluorescein, diacetoxymethyl difluorofluorescein, 7-acetyl, 7-hydroxycoumarin, 7-acetyl, 7-hydroxy, 4-methylcoumarin, 7-acetyl, 7-hydroxy, 3-methylcoumarin, 7-acetoxymethyl, 7-hydroxycoumarin, 7-acetoxymethyl, 7-hydroxy, 4-methylcoumarin, 7-acetoxymethyl, 7-hydroxy, 3-methylcoumarin can be used.

In FIG. 2B, the transformation is achieved by an exogenous “direct pathway” staining protocol, wherein the pathway using a first exogenously supplied molecule 204 is referred to as the “direct pathway.” In some embodiments, a second labeling molecule 206 is transformed into a second stain molecule 208. Unlike the first labeling molecule 200, the second labeling molecule 206 is not readily cell permeable. In some embodiments, the second labeling molecule 206 is non-fluorescent or weakly fluorescent, whereas the second stain molecule 208 is moderately or brightly fluorescent. The second labeling molecule 206 is non-toxic. The second labeling molecule 206 also preferably does not interfere with important cellular pathways and processes. The second stain molecule 208 is also non-toxic and does not interfere with important cellular pathways and processes. In some embodiments, the transformation is mediated by the first exogenously supplied molecule 204. The first exogenously supplied molecule 204 binds selectively to the desired cell 104. In some embodiments, the first exogenously supplied molecule 204 is an antibody-enzyme conjugate, in which the antibody binds specifically to the desired cell 104. In some embodiments, the enzyme within the first exogenously supplied molecule 204 catalyzes the transformation of the second labeling molecule 206 to the second stain molecule 208. In some embodiments, the exogenous “direct pathway” staining protocol follows similar protocols as that of the standard direct ELISA. The enzyme portion of 204 may be horseradish peroxidase (HRP), alkaline phosphatase (AP), or luciferase. In some embodiments, labeling molecules used with HRP can include, but are not limited to, p-Nitrophenyl Phosphate and 3,3′,5,5′-tetramethylbenzidine, labeling molecules used with AP can include, but are not limited to, Attophos and 4-Methylumbelliferyl phosphate, labeling molecules used with luciferase can include, but are not limited to, D-luciferin. In some embodiments, one or more of the following can also be used: 3,3′,5,5′-Tetramethylbenzidine, 2,2′-azino-bis(3-ethylbenzothiazoline-6-sulphonic acid), 4-chloro-1-napthol, o-Phenylenediamine, Nitroblue Tetrazolium, 5-bromo-4-chloro-3′-indolyphosphate p-toluidine.

In FIG. 2C, the transformation is achieved by an exogenous “indirect pathway” staining protocol, wherein the pathway using a second exogenously supplied molecule 210 is referred to as the “indirect pathway.” The second exogenously supplied molecule 210 binds selectively to the desired cell 104. In some embodiments, the third exogenously supplied molecule 212 is an antibody-enzyme conjugate that can bind to the second exogenously supplied molecule 210 selectively. The third exogenously supplied molecule 212 catalyzes the transformation of a third labeling molecule 214 to a third stain molecule 216. In some embodiments, the third labeling molecule 214 is non-fluorescent or weakly fluorescent. In some embodiments, the third stain molecule 216 is moderately or brightly fluorescent. The third labeling molecule 214 is also non-toxic and does not interfere with important cellular pathways and processes. Unlike the first labeling molecule 200, the third labeling molecule 214 is not readily cell permeable. In addition, the third labeling molecule 214 may be the same as the second labeling molecule 206. The third stain molecule 216 is also non-toxic and does not interfere with important cellular pathways and processes. In some embodiments, the exogenous “indirect pathway” staining protocol follows similar protocols as that of the standard indirect ELISA. The third exogenously supplied molecule 212 may be goat anti-mouse IgG:HRP or goat anti-mouse IgG:AP. As noted above, IgG:HRP can be matched with HRP molecules (e.g., p-Nitrophenyl Phosphate and 3,3′,5,5′-tetramethylbenzidine) and IgG:AP can be matched with AP molecules (e.g., Attophos and 4-Methylumbelliferyl phosphate), as well as other labeling molecules listed above.

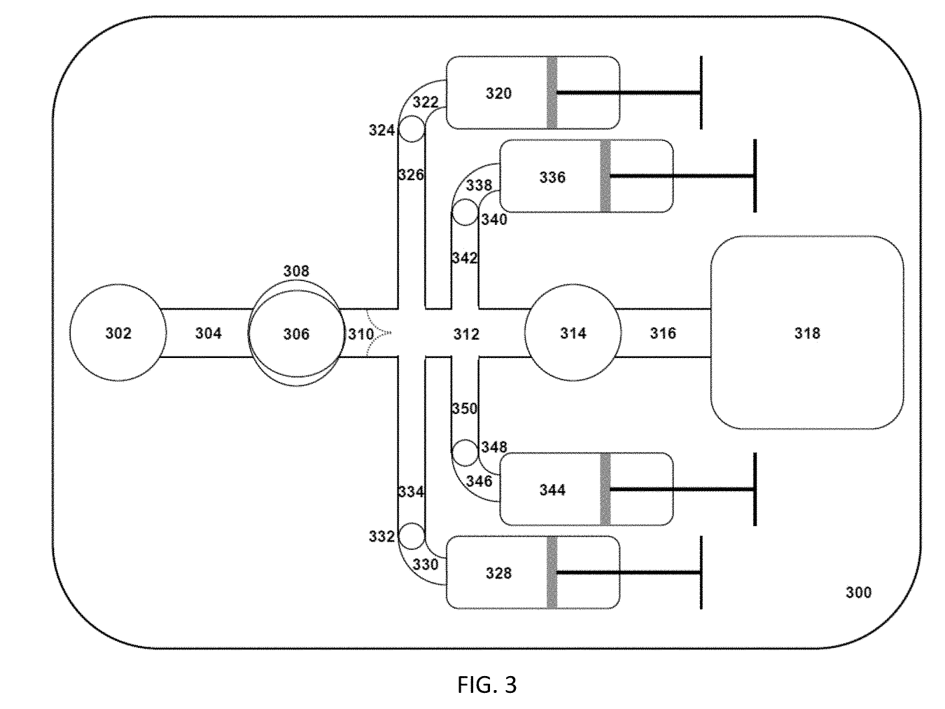

FIG. 3 illustrates the design of the microfluidic chip 300 in accordance with some embodiments. As illustrated, a body fluid (e.g. blood) is applied to the microfluidic chip 300 and is collected in a first reservoir 302. In some embodiments, the body fluid is treated with an anticoagulant before it enters into the first reservoir 302. In some embodiments, the body fluid is anticoagulated when it flows through a first channel 304 that is coated with the anticoagulant. In some embodiments, the anticoagulant comprises K₂EDTA or fluoride-oxalate. In some embodiments, the anticoagulant comprises heparin but may also comprise citrate.

In some embodiments, the volume of the body fluid is controlled by obtaining a fixed amount of the body fluid before applying the body fluid to the first reservoir 302. In some embodiments, the volume of the body fluid is controlled by a second reservoir 306 that has a specified fluid capacity (5-50 μL), and that features a gutter 308 that fills with the excess body fluid. In some embodiments, fluid can be metered using a microcapillary with a defined fill volume, or using a metered check-valve.

The flow of the body fluid is controlled by a mechanism 310 that allows the body fluid to flow unidirectionally from the first reservoir 302, through a second channel 312, and into the capture chamber 314. In some embodiments, the mechanism 310 comprises a one-way valve. The body fluid flows from the capture chamber 314 through a third channel 316 into a waste collection reservoir 318.

In some embodiments, a wash solution, consisting of PBS, is injected from a first container 320 through a first plastic tubing 322, a first microfluidic port 324, and a fourth channel 326 into the second channel 312 of the microfluidic chip 300. In some embodiments, the wash solution washes the residual body fluid remaining in the second channel 312 into the capture chamber 314 and the wash solution further washes away any undesired components of the body fluid that have not been immobilized to the surface of the capture chamber 314. The wash solution further flushes the undesired components of the body fluid into the waste collection reservoir 318. In some embodiments, the wash solution is not necessary to wash away the undesired components of the body fluid. In some embodiments, the first container 320 comprises a syringe that is actuated to push out the wash solution.

In some embodiments where the endogenous staining protocol is desired, the first labeling solution 200 is injected from a second container 328 through a second plastic tubing 330, a second microfluidic port 332, and a fifth channel 334 into the second channel 312 of the microfluidic chip 300. The first labeling solution 200 reacts with immobilized substance in the capture chamber 314 as described above with respect to FIG. 2. In some embodiments, the second container 328 comprises a syringe that is actuated to push out the first labeling solution 200.

In some embodiments, where the exogenous “direct pathway” staining protocol is desired, the first exogenously supplied molecule solution 204 is injected from a third container 344 through a third plastic tubing 346, a third microfluidic port 348, and a sixth channel 350 into the second channel 312 of the microfluidic chip 300. The first exogenously supplied molecule solution 204 interacts with immobilized substance in the capture chamber 314 as described above with respect to FIG. 2. In some embodiments, the third container 344 comprises a syringe that is actuated to push out the solution 204.

In some embodiments, after the first exogenously supplied molecule solution 204 interacts with immobilized substance in the capture chamber 314 as described above with respect to FIG. 2, the wash solution, consisting of PBS, is injected from the first container 320 through a first plastic tubing 322, a first microfluidic port 324, and a fourth channel 326 into the second channel 312 of the microfluidic chip 300. In some embodiments, the wash solution washes the residual first exogenously supplied molecule solution 204 remaining in the second channel 312 into the capture chamber 314 and the wash solution further washes away any first exogenously supplied molecule solution 204 that have not been immobilized to the surface of the capture chamber 314. In some embodiments, the wash solution further flushes the undesired first exogenously supplied molecule solution 204 into the waste collection reservoir 318. In some embodiments, the wash solution is not necessary to wash away the first exogenously supplied molecule solution 204.

In some embodiments, a second labeling solution 206 is injected from the second container 328 through a second plastic tubing 330, a second microfluidic port 332, and a fifth channel 334 into the second channel 312 of the microfluidic chip 300. In some embodiments, the second labeling solution 206 reacts with immobilized substance in the capture chamber 314 as described above with respect to FIG. 2.

In some embodiments where the exogenous “indirect pathway” staining protocol is desired, a second exogenously supplied molecule solution 210 is injected from a fourth container 336 through a fourth plastic tubing 338, a fourth microfluidic port 340, and a seventh channel 342 into the second channel 312 of the microfluidic chip 300. In some embodiments, the second exogenously supplied molecule solution 210 interacts with the immobilized substance in the capture chamber 314 as described above with respect to FIG. 2. In some embodiments, the fourth container 336 comprises a syringe that is actuated to push out the solution 210.

In some embodiments, after the second exogenously supplied molecule solution 210 interacts with the immobilized substance in the capture chamber 314 as described above with respect to FIG. 2, the wash solution, consisting of PBS, is injected from a first container 320 through a first plastic tubing 322, a first microfluidic port 324, and a fourth channel 326 into the second channel 312 of the microfluidic chip 300. In some embodiments, the wash solution washes the residual second exogenously supplied molecule solution 210 remaining in the second channel 312 into the capture chamber 314 and the wash solution further washes away any second exogenously supplied molecule solution 210 that have not been immobilized to the surface of the capture chamber 314. In some embodiments, the wash solution further flushes the undesired second exogenously supplied molecule solution 210 into the waste collection reservoir 318. In some embodiments, the wash solution is not necessary to wash away the second exogenously supplied molecule solution 210.

In some embodiments, after the second exogenously supplied molecule solution 210 interacts with the immobilized substance in the capture chamber 314 as described above with respect to FIG. 2, a third exogenously supplied molecule solution 212 is injected from the third container 344 through the third plastic tubing 346, the third microfluidic port 348, and the sixth channel 350 into the second channel 312 of the microfluidic device. The third exogenously supplied molecule solution 212 interacts with the second exogenously supplied molecules 210 in the capture chamber 314 as described above with respect to FIG. 2.

In some embodiments, after the third exogenously supplied molecule solution 212 interacts with the second exogenously supplied molecules 210 in the capture chamber 314 as described above with respect to FIG. 2, the wash solution, consisting of PBS, is injected from the first container 320 through a first plastic tubing 322, a first microfluidic port 324, and a fourth channel 326 into the second channel 312 of the microfluidic chip 300. The wash solution washes the residual third exogenously supplied molecule solution 212 remaining in the second channel 312 into the capture chamber 314 and the wash solution further washes away any third exogenously supplied molecule solution 212 that have not been immobilized to the surface of the capture chamber 314. In some embodiments, the wash solution further flushes the undesired third exogenously supplied molecule solution 212 into the waste collection reservoir 318. In some embodiments, the wash solution is not necessary to wash away the third exogenously supplied molecule solution 212.

In some embodiments, after the third exogenously supplied molecule solution 212 interacts with the second exogenously supplied molecules 210 in the capture chamber 314 as described above with respect to FIG. 2, the third labeling solution 214 is injected from the second container 328 through a second plastic tubing 330, a second microfluidic port 332, and a fifth channel 334 into the second channel 312 of the microfluidic chip 300. In some embodiments, the third labeling solution 214 reacts with immobilized substance in the capture chamber 314 as described above with respect to FIG. 2.

In some embodiments, the microfluidic chip 300 comprises a polymeric material, such as PDMS, in the usual manner with soft lithography. In some embodiments, the containers 320, 328, 336, and 344 are attached to the microfluidic chip 300 using glue. In some embodiments, the plastic tubings 322, 330, 338, and 346 are attached to the nozzles of the containers 320, 328, 336, and 344 and to the microfluidic ports 324, 332, 340, and 348, respectively, with a tight fit and some reinforcement. The microfluidic ports 324, 332, 340, and 348 are openings on the microfluidic chip 300 that allow matters inside the containers 320, 328, 336, and 344 to flow through the plastic tubings 322, 330, 338, and 346 into the microfluidic chip 300.

In some embodiments, the wash solution may be contained in a plurality of containers rather than a single container.

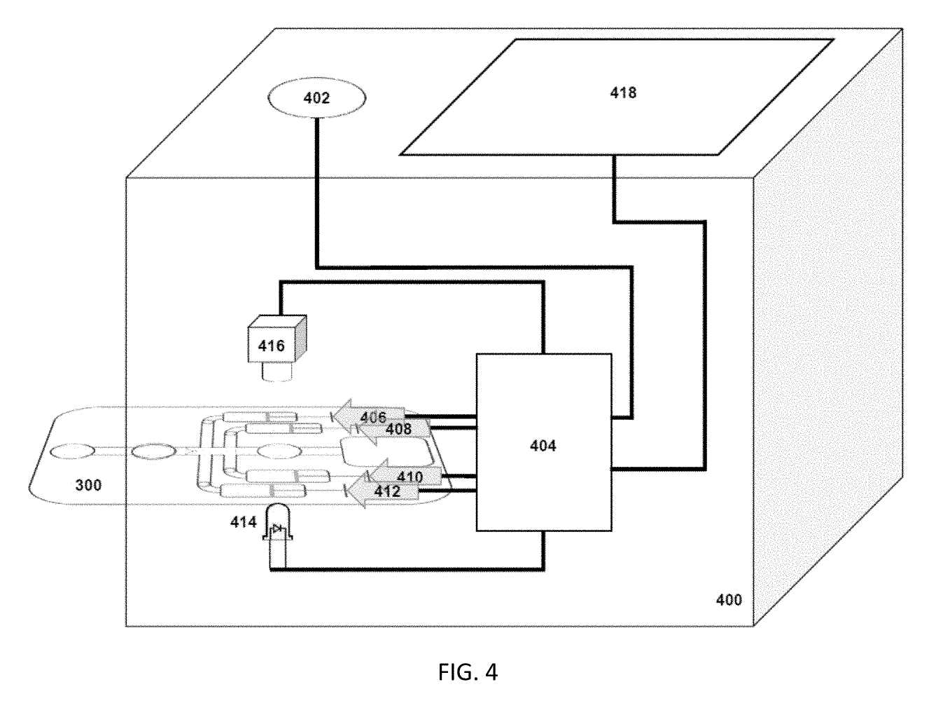

FIG. 4 illustrates an electronic reader 400 with the microfluidic chip 300 placed within. In some embodiments, the electronic reader 400 is turned on by pressing a button 402. In some embodiments, the electronic reader 400 detects the microfluidic chip 300 when a first container controller 406 connects with the first container 320, a second container controller 412 with the second container 328, a third container controller 410 with the third container 344, and a fourth container controller 408 with the fourth container 336. The presence of the microfluidic chip 300 may be acknowledged by activation of a light source 414 and an optical detection unit 416. Entry of the body fluid into the capture chamber also may be recognized by the light source 414 and the optical detection unit 416.

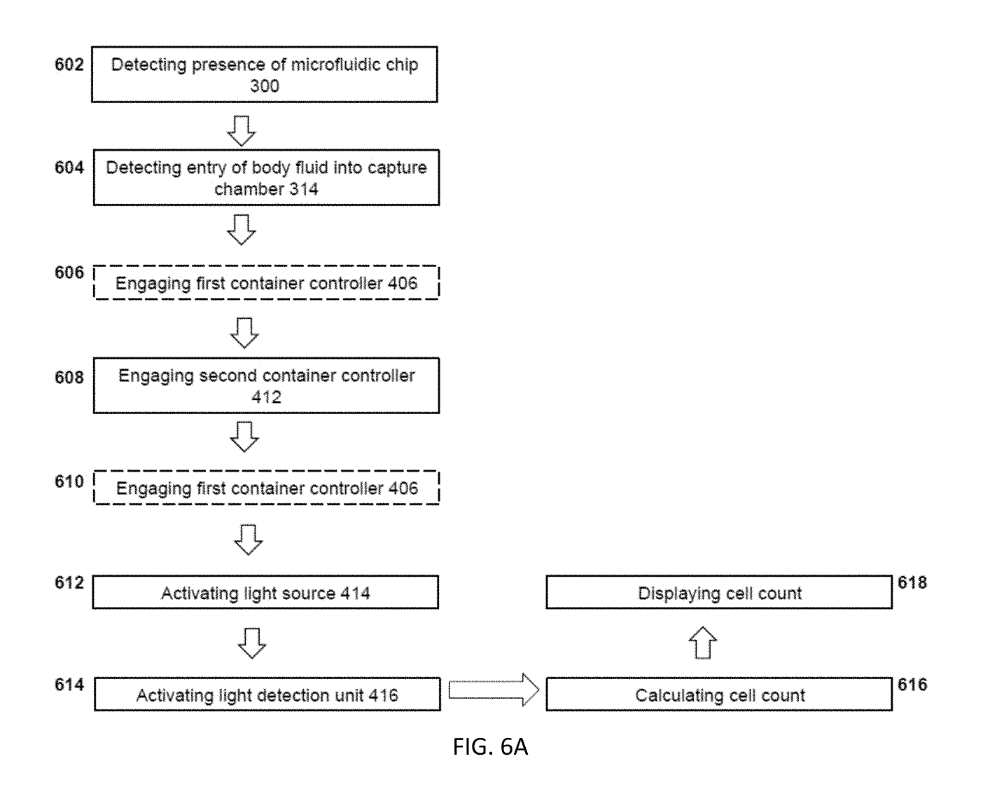

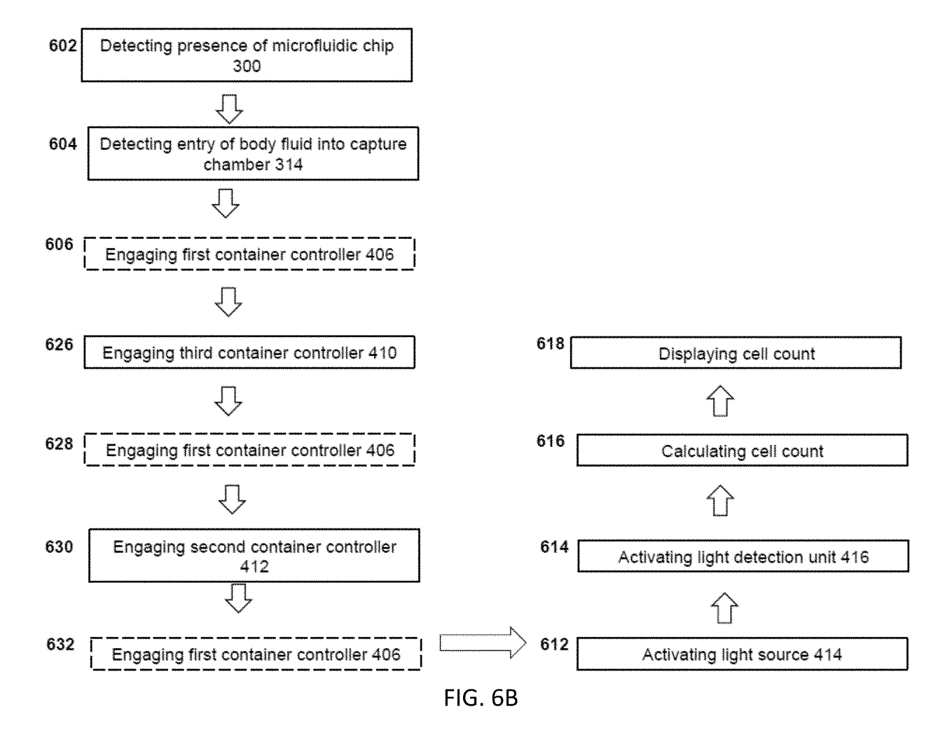

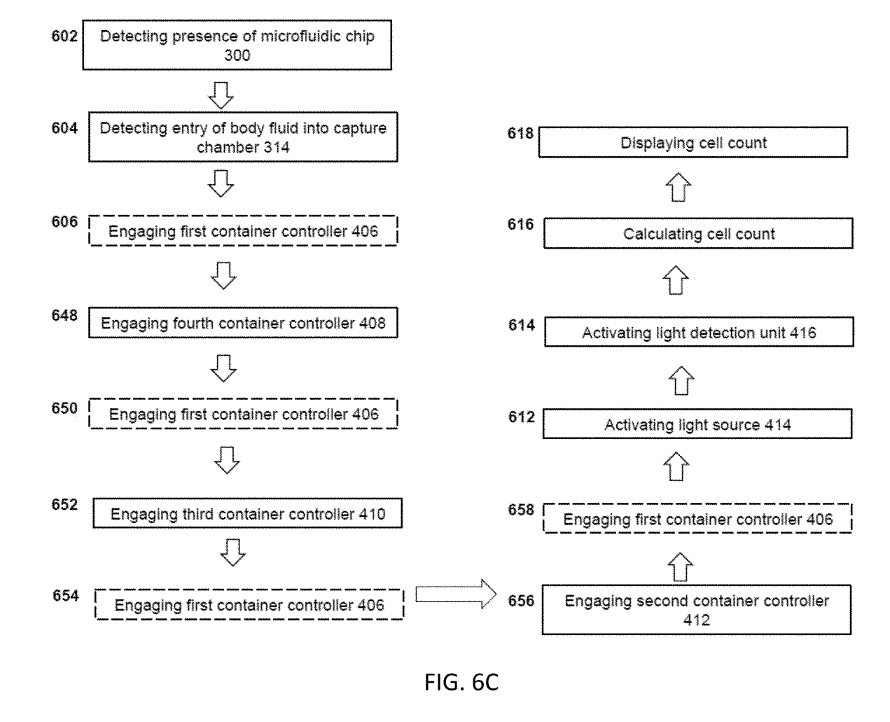

In some embodiments, the microprocessor 404 processes information input and coordinates further commands and functions as described more fully below with respect to FIG. 6.

In some embodiments where the endogenous staining protocol is desired, the microprocessor 404 engages the second container controller 412 to release the first labeling molecule solution 200 from the second container 328 after the body fluid is immobilized in the capture chamber 314. The microprocessor 404 engages the first container controller 406 to release the wash solution from the first container 320 after the body fluid is immobilized in the capture chamber 314 and before the microprocessor 404 engages the second container controller 412. In some embodiments, the microprocessor 404 does not engage the first container controller 406 to release the wash solution from the first container 320.

In some embodiments where the exogenous “direct pathway” protocol is desired, the microprocessor 404 engages the third container controller 410 to release the first exogenously supplied molecule solution 204 from the third container 344 after the body fluid is immobilized in the capture chamber 314. In some embodiments, the microprocessor 404 further engages the second container controller 412 to release the second labeling solution 206 from the second container 328 after the first exogenously supplied molecule solution 204 is immobilized in the capture chamber 314.

In some embodiments where the exogenous “direct pathway” protocol is desired, the microprocessor 404 engages the first container controller 406 to release the wash solution from the first container 320 after the body fluid is immobilized in the capture chamber 314 and before the microprocessor 404 engages the third container controller 410. In some embodiments, the microprocessor 404 does not engage the first container controller 406 to release the wash solution from the first container 320. The microprocessor 404 further engages the first container controller 406 to release the wash solution from the first container 320 after the first exogenously supplied molecule solution 204 is immobilized in the capture chamber 314 and before the microprocessor 404 further engages the second container controller 412. In some embodiments, the microprocessor 404 does not engage the first container controller 406 to release the wash solution from the first container 320.

In some embodiments where the exogenous “indirect pathway” protocol is desired, the microprocessor 404 engages the fourth container controller 408 to release the second exogenously supplied molecule solution 210 from the fourth container 336 after the body fluid is immobilized in the capture chamber 314. In some embodiments, the microprocessor 404 further engages the third container controller 410 to release the third exogenously supplied molecule solution 212 from the third container 344 after the second exogenously supplied molecule solution 210 is immobilized in the capture chamber 314. The microprocessor 404 then engages the second container controller 412 to release the third labeling solution from the second container 328 after the second exogenously supplied molecule solution 210 is immobilized in the capture chamber 314. The microprocessor 404 engages the first container controller 406 to release the wash solution from the first container 320 after the body fluid is immobilized in the capture chamber 314 and before the microprocessor 404 engages the second container controller 412. In some embodiments, the microprocessor 404 does not engage the first container controller 406 to release the wash solution from the first container 320. Also, the microprocessor 404 engages the first container controller 406 to release the wash solution from the first container 320 after the second exogenously supplied molecule solution 210 is immobilized in the capture chamber 314 and before the microprocessor 404 further engages the third container controller 410. In some embodiments, the microprocessor 404 does not engage the first container controller 406 to release the wash solution from the first container 320. The microprocessor 404 further engages the first container controller 406 to release the wash solution from the first container 320 after the third exogenously supplied molecule solution 212 is immobilized in the capture chamber 314 and before the microprocessor 404 then engages the second container controller 412. In some embodiments, the microprocessor 404 does not engage the first container controller 406 to release the wash solution from the first container 320.

The microprocessor activates the light source 414 and the optical detection unit 416. In some embodiments, the light source 414 emits light that corresponds to the photochemical properties of the labeling molecule 200, 206 and/or 214. In some embodiments, the optical detection unit 416 detects optical signals that correspond to the photochemical properties of the labeling molecule 200, 206 and/or 214.

In some embodiments, the light source 414 comprises a narrow-spectrum light source that emits light corresponding to the photochemical properties of the labeling molecule 200, 206 and/or 214. In some embodiments, the light source 414 comprises a broad-spectrum light source with a first optical filter that allows only light corresponding to the photochemical properties of the labeling molecule 200, 206 and/or 214 to pass through.

In some embodiments, the optical detection unit 416 comprises a detector that detects the optical signals that correspond to the photochemical properties of the labeling molecule 200, 206, and/or 214. In some embodiments, the optical detection unit 416 comprises a broad-spectrum light detector with a second optical filter that allows light that that corresponds to the photochemical properties of the labeling molecule 200, 206, and/or 214 to pass through.

Once the optical detection unit 416 detects the optical signal, the microprocessor 404 applies the measured optical signal to a pre-determined algorithm to determine the cell count. In some embodiments, the pre-determined algorithm is derived from clinical trials using this system in patients with known cell counts. For example, in some embodiments, the algorithm may simply compare the optical signal to a curve derived from the signals corresponding to known cell counts. The microprocessor 404 then displays the cell count on a screen 418.

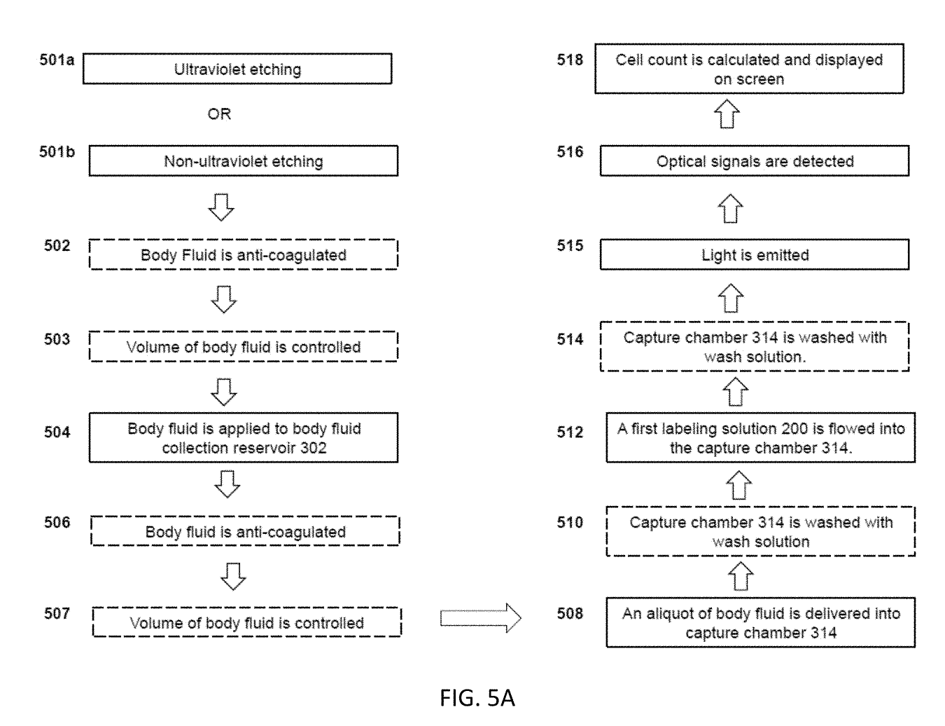

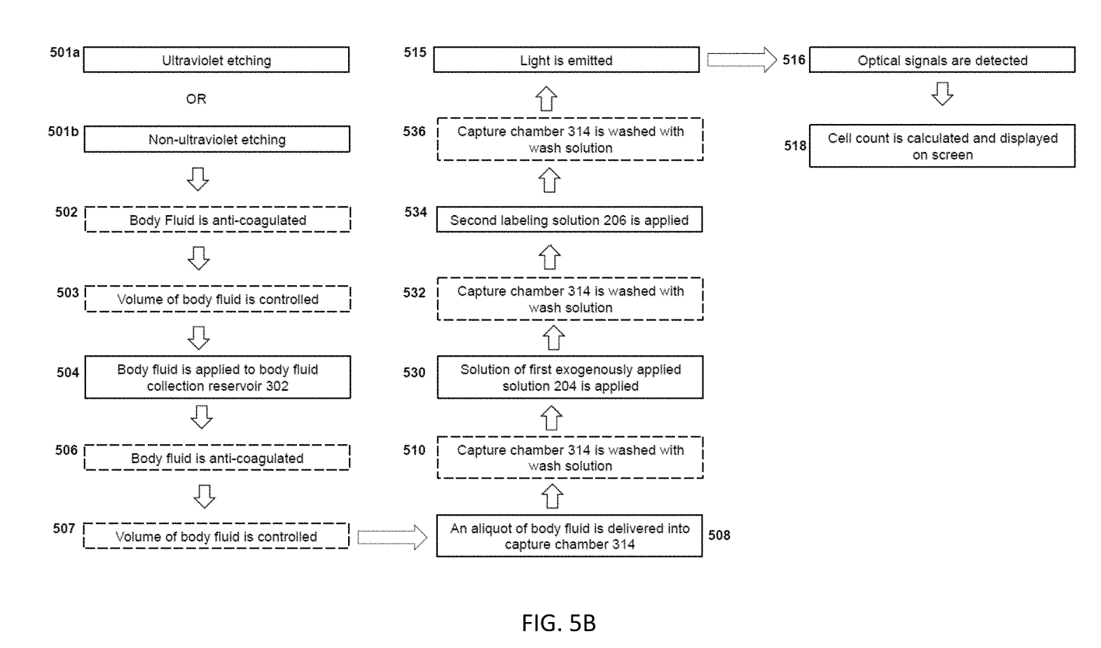

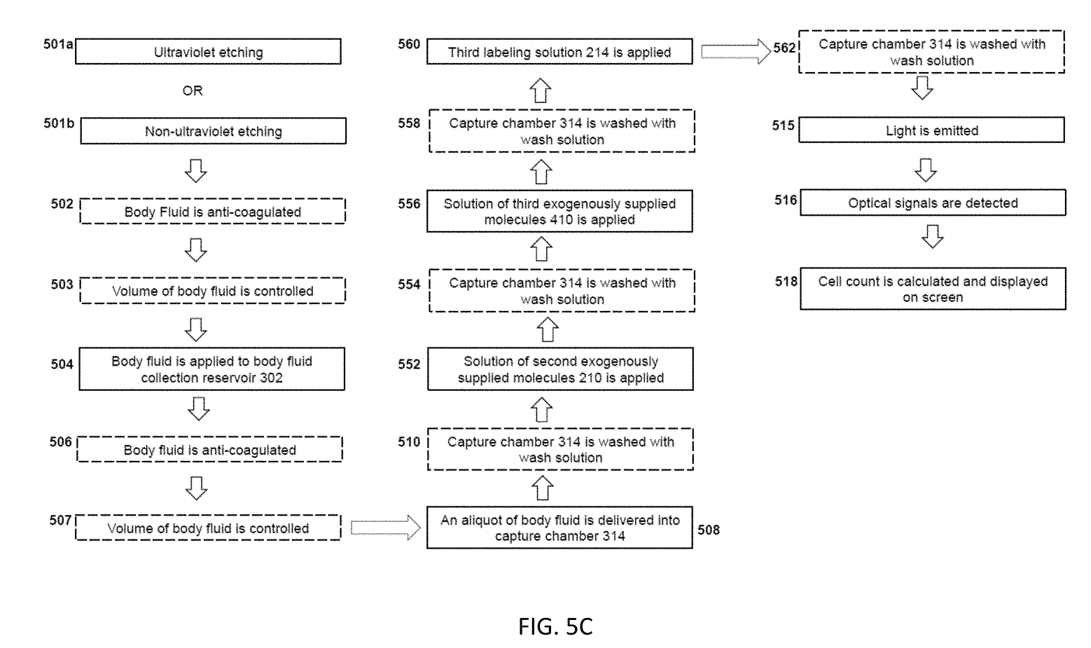

FIG. 5A is a simplified flowchart of a method for counting desired cells in a body fluid where the endogenous staining protocol is desired. Boxes drawn in dotted lines are optional steps.

In Step 501a, the solid scaffold 100 of the capture chamber 314 is treated with ultraviolet radiation and then attached with the recognition molecules 102. In some embodiments, the solid scaffold 100 comprises PMMA and the recognition molecule 102 is an antibody or protein. In some embodiments, the surface of the solid scaffold 100 is activated by irradiation with UV light (254 nm with 15 mW cm⁻² fluence for 10 min) The surface of the solid scaffold 100 is then further activated by treating with an activating solution of 25 mM 1-ethyl-3-[3-dimethylaminopropyl] carbodiimide hydrochloride and 50 mM N-hydroxysuccinimide in 150 mM 2-(4-morpholino)-ethane sulfonic acid at pH 6 for 1 hour. Then the activating solution is removed and a solution of the recognition molecule 102 (1 mg/mL) is applied to the solid scaffold 100 in 150 mM phosphate buffered saline (PBS) at pH 7.4 for 4 hours. Afterwards the solution of the recognition molecule 102 is removed, and the solid scaffold 100 is rinsed with PBS. In some embodiments, this method is based on that described by Adams, et al. (Adams, et al. J. Am Chem Soc. 2008).

In Step 501b, which is an alternative to the Step 501a, the solid scaffold 100 of the capture chamber 314 is attached with the linker 106, then the recognition molecules 102. In some embodiments, the solid scaffold 100 comprises PDMS and the recognition molecule 102 is an antibody or protein. In some embodiments, the linker 106 comprises one chemical moiety or a linear arrangement of a plurality of other chemical moieties. In some embodiments, the linker 106 comprises three moieties: 3-mercaptopropyl trimethoxysilane, N-y-maleimidobutyryloxy succinimide ester (GMBS), and NeutrAvidin. The solid scaffold 100 is first treated with a 4% (v/v) solution of 3-mercaptopropyl trimethoxysilane in ethanol at room temperature for 30 min. The solution of 3-mercaptopropyl trimethoxysilane is removed by flushing the solid scaffold 100 with ethanol. The solid scaffold 100 is then treated with a 0.25 mg/mL solution of GMBS in dimethylsulfoxide (DMSO) at room temperature for 15 min. Then the solution of GMBS is removed by flushing 100 with ethanol. The solid scaffold 100 is then treated with a solution of 0.1% (v/v) NeutrAvidin (reconstituted from lyophilized powder as per the manufacturer's instructions) in PBS for 18 h at 4° C. The solution of NeutrAvidin is removed by flushing the solid scaffold 100 with PBS. The solid scaffold 100 is then flushed with a solution of 0.1% bovine serum albumin (BSA) in PBS. The solid scaffold 100 is then treated with a 1 μg/mL solution of 102 in PBS at room temperature for 2 hours. Finally, the solution of the recognition molecule 102 is removed by flushing the solid scaffold 100 with PBS. In some embodiments, this method is based on that described by Murthy, et al (Murthy, et al. Langmuir 2004).

In optional Step 502, the body fluid is treated with an anticoagulant before it enters into the first reservoir 302 so that the body fluid will not coagulate when it enters the capture chamber 314. In some embodiments, the anticoagulant comprises K₂EDTA. In some embodiments, the anticoagulant comprises heparin or citrate. In optional Step 503, the volume of the body fluid is controlled by obtaining a fixed amount of the body fluid before applying the body fluid to the first reservoir 302.

In Step 504, the body fluid is applied to the microfluidic chip 300 and is collected in the first reservoir 302.

In optional Step 506, the body fluid is anticoagulated when it flows through the first channel 304 that is coated with the anticoagulant. In some embodiments, the anticoagulant comprises K₂EDTA, heparin, or citrate. In optional Step 507, the volume of the body fluid is controlled by a second reservoir 306 that has a specified fluid capacity (5-50 μL), and that features a gutter 308 that fills with the excess body fluid.

In Step 508, an aliquot of body fluid is delivered into the capture chamber 314 so that the capture chamber 314 can immobilize the desired cells within the body fluid. In some embodiments, the desired cells are neutrophils.

In optional Step 510, the wash solution is applied to the capture chamber 314 to remove the residual body fluid from the capture chamber 314. In some embodiments, the wash solution comprises PBS.

In Step 512, the first labeling solution 200 is delivered to the capture chamber 314 to react with immobilized desired cells in the capture chamber 314. In some embodiments, the first labeling molecule 200 is non-fluorescent or weakly fluorescent. The first labeling molecule 200 is non-toxic and does not interfere with important cellular pathways and processes. The first labeling molecule 200 is preferably sufficiently cell-permeable. In some embodiments, the first labeling molecule 200 may be fluorescein diacetate (FDA) and diffuses into the desired cells and reacts with the enzymes of the desired cells 104.

In optional Step 514, the wash solution is applied to the capture chamber 314 to remove the residual labeling solution 200 from the capture chamber 314, especially when the labeling solution 200 is to certain extent fluorescent. In some embodiments, the wash solution comprises PBS.

In Step 515, light that corresponds to the photochemical properties of the labeling molecule 200 is emitted by the light source 414 to excite the stain solution 202. In some embodiments, the light source 414 comprises a narrow-spectrum light source that emits light that corresponds to the photochemical properties of the labeling molecule 200. In some embodiments, the light source 414 comprises a broad-spectrum light source with a first optical filter that allows only light that that corresponds to the photochemical properties of the labeling molecule 200 to pass through. In Step 516, the optical signals that that corresponds to the photochemical properties of the labeling molecule 200 are detected by the optical detection unit 416 to facilitate the further calculation of the cell count. In some embodiments, the optical detection unit 416 comprises a detector that detects the optical signals that that corresponds to the photochemical properties of the labeling molecule 200. In some embodiments, the optical detection unit 416 comprises a broad-spectrum light detector with a second optical filter that allows light that that corresponds to the photochemical properties of the labeling molecule 200 to pass through.

In step 518, the cell count is calculated based on the pre-determined algorithm and displayed on the screen 418. In some embodiments, the pre-determined algorithm is derived from clinical trials using this system in patients with known cell counts.

FIG. 5B is a simplified flowchart of a method for counting desired cells in a body fluid where the exogenous “direct pathway” protocol is desired. Boxes drawn in dotted lines are optional steps. In FIG. 5B, steps 501-510 are the same as set forth above with respect to FIG. 5A. However, at Step 530, the first exogenously supplied molecule solution 204 is delivered to the capture chamber 314 to react with immobilized desired cells in the capture chamber 314. The first exogenously supplied molecule 204 is an antibody linked to alkaline phosphatase (AP).

In optional Step 532, the wash solution is applied to the capture chamber 314 to remove the residual first exogenously supplied molecule solution 204 from the capture chamber 314. In some embodiments, the wash solution comprises PBS.

In Step 534, the second labeling solution 206 is delivered to the capture chamber 314 to react with immobilized first exogenously supplied molecules 204 in the capture chamber 314. In some embodiments, the second labeling solution 206 is non-fluorescent or weakly fluorescent and is non-toxic. The second labeling solution 206 preferably does not interfere with important cellular pathways and processes. In some embodiments, the second labeling 206 may be AttoPhos, or 4-methylumbelliferone.

In optional Step 536, the wash solution is applied to the capture chamber 314 to remove the residual second labeling solution 206 from the capture chamber 314, especially when the second labeling solution 206 is to certain extent fluorescent. In some embodiments, the wash solution comprises PBS.

Steps 515-518 as described above with respect to FIG. 5A are then applied to the stain solution 208 for emission of the light, detection of the fluorescence and determination of the cell count.

FIG. 5C is a simplified flowchart of a method for counting desired cells in a body fluid where the exogenous “indirect pathway” protocol is desired. Boxes drawn in dotted lines are optional steps. In FIG. 5C, steps 501-510 are the same as set forth above with respect to FIG. 5A. However, at Step 552, the second exogenously supplied molecule solution 210 is applied to the capture chamber 314 to react with immobilized desired cells in the capture chamber 314. The second exogenously supplied molecule 210 is an antibody that binds selectively to the desired cell 104.

In optional Step 554, the wash solution is applied to the capture chamber 314 to remove the second exogenously supplied molecule solution 210 from the capture chamber 314. In some embodiments, the wash solution comprises PBS.

In Step 556, the third exogenously supplied molecule solution 212 is delivered to the capture chamber 314 to react with the immobilized second exogenously supplied molecules 210 in the capture chamber 314. The third exogenously supplied molecule 212 is an appropriate secondary antibody linked to alkaline phosphatase.

In optional Step 558, the wash solution is applied to the capture chamber 314 to remove the third exogenously supplied molecule solution 212 from the capture chamber 314. In some embodiments, the wash solution comprises PBS.

In Step 560, the third labeling solution 214 is delivered to the capture chamber 314 to react with the immobilized third exogenously supplied molecules 212 in the capture chamber 314. In some embodiments, the third exogenously supplied molecules 212 are non-fluorescent or weakly fluorescent. In some embodiments, the third exogenously supplied molecules 212 are non-toxic. In some embodiments, the third exogenously supplied molecules 212 do not interfere with important cellular pathways and processes. The third labeling 214 may be AttoPhos, or 4-methylumbelliferone.

In optional Step 562, the wash solution is applied to the capture chamber 314 to remove the residual third labeling solution 214 from the capture chamber 314, especially when the third labeling solution 214 is to certain extent fluorescent. In some embodiments, the wash solution comprises PBS.

Steps 515-518 as described above with respect to FIG. 5A are then applied to the stain solution 216 for emission of the light, detection of the fluorescence and determination of the cell count.

FIG. 6A is a simplified flowchart describing the workflow of the microprocessor 404 where the endogenous staining protocol is desired. Boxes drawn in dotted lines are optional steps.

In Step 602, the microprocessor 404 detects the presence of the microfluidic chip 300. In some embodiments, the electronic reader 400 detects the microfluidic chip 300 when a first container controllers 406 connects with the first container 320, a second container controller 412 with the second container 328, a third container controller 410 with the third container 344, and a fourth container controller 408 with the fourth container 336. The presence of the microfluidic chip 300 may be confirmed by activation of a light source 414 and an optical detection unit 416.

In Step 604, the microprocessor 404 detects the entry of the body fluid into the capture chamber 314. In some embodiments, entry of the body fluid into the capture chamber is also identified by the light source 414 and the optical detection unit 416.

In optional Step 606, the microprocessor 404 engages the first container controller 406 to release wash from the first container 320 to remove the residual body fluid from the capture chamber 314.

In Step 608, the microprocessor 404 engages the second container controller 412 to release the first labeling 200 from the second container 328 so that the first labeling 200 can react immobilized desired cells in the capture chamber 314.

In optional Step 610, the microprocessor 404 engages the first container controller 406 to release wash from the first container 320 to remove the residual labeling solution 200 from the capture chamber 314, especially when the labeling solution 200 is to certain extent fluorescent. In some embodiments, the wash solution comprises PBS.

In Step 612, the microprocessor 404 activates the light source 414 to excite the stain solution 202.

In Step 614, the microprocessor 404 activates the optical detection unit 416 to detect the optical signal that corresponds to the photochemical properties of the labeling molecule 200.

In Step 616, the microprocessor 404 calculates the cell count based on the pre-determined algorithm.

In Step 618, the microprocessor 404 displays the cell count on the screen 418.

FIG. 6B is a simplified flowchart describing the workflow of the microprocessor 404 where the exogenous “direct pathway” protocol is desired. Boxes drawn in dotted lines are optional steps. In FIG. 6B, steps 602-606 are the same as set forth above with respect to FIG. 6A. However, at Step 626, the microprocessor 404 engages the third container controller 410 to release the first exogenously supplied molecule solution 204 from the third container 344, such that the first exogenously supplied molecule solution 204 reacts with immobilized desired cells in the capture chamber 314.

In optional Step 628, the microprocessor 404 engages the first container controller 406 to release wash from the first container 320 to remove the residual first exogenously supplied molecule solution 204 from the capture chamber 314.

In Step 630, the microprocessor 404 engages the second container controller 412 to release the second labeling 206 from the second container 328, such that the second labeling solution 206 reacts with immobilized first exogenously supplied molecules 204 in the capture chamber 314.

In optional Step 632, the microprocessor 404 engages the first container controller 406 to release wash from the first container 320 to remove the residual second labeling solution 206 from the capture chamber 314, especially when the second labeling solution 206 is to certain extent fluorescent. In some embodiments, the wash solution comprises PBS.

The microprocessor 404 then activates the light source 414 to excite the stain solution 208 and activates the optical detection unit 416 to detect the optical signal that that corresponds to the photochemical properties of the labeling molecule 206 as described above with respect to steps 612 and 614 and calculates and displays the cell count as described above with respect to steps 616 and 618.

FIG. 6C is a simplified flowchart describing the workflow of the microprocessor 404 where the exogenous “indirect pathway” protocol is desired. Boxes drawn in dotted lines are optional steps. In FIG. 6B, steps 602-606 are the same as set forth above with respect to FIG. 6A. However, at Step 648, the microprocessor 404 engages the fourth container controller 408 to release the second exogenously supplied molecule solution 210 from the fourth container 336, such that the second exogenously supplied molecule solution 210 reacts with immobilized desired cells 104 in the capture chamber 314.

In optional Step 650, the microprocessor 404 engages the first container controller 406 to release wash from the first container 320 to remove the residual second exogenously supplied molecule solution 210 from the capture chamber 314.

In Step 652, the microprocessor 404 engages the third container controller 410 to release the third exogenously supplied molecule solution 212 from the third container 344, such that the third exogenously supplied molecule solution 212 reacts with the immobilized second exogenously supplied molecules 210 in the capture chamber 314.

In optional Step 654, the microprocessor 404 engages the first container controller 406 to release wash from the first container 320 to remove the residual third exogenously supplied molecule solution 212 from the capture chamber 314.

In Step 656, the microprocessor 404 engages the second container controller 412 to release the third labeling solution 214 from the second container 328, such that the third labeling solution 214 reacts with the immobilized third exogenously supplied molecules 212 in the capture chamber 314.

In optional Step 658, the microprocessor 404 engages the first container controller 406 to release wash from the first container 320 to remove the residual labeling solution 214 from the capture chamber 314, especially when the labeling solution 214 is to certain extent fluorescent. In some embodiments, the wash solution comprises PBS.

The microprocessor 404 then activates the light source 414 to excite the stain solution 216 and activates the optical detection unit 416 to detect the optical signal that that corresponds to the photochemical properties of the labeling molecule 214 as described above with respect to steps 612 and 614 and calculates and displays the cell count as described above with respect to steps 616 and 618.

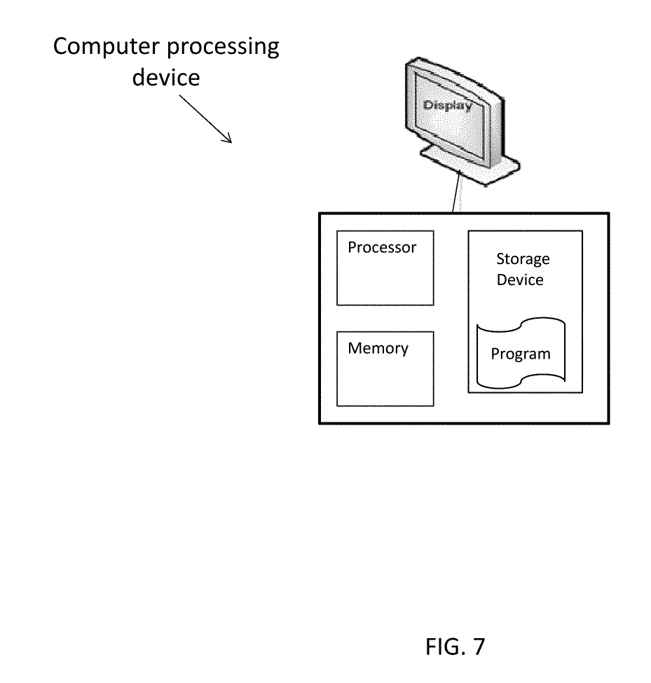

FIG. 7 shows, by way of example, a diagram of a typical processing architecture, which may be used in connection with the methods and systems of the present disclosure. A computer processing device can be coupled to a display for graphical output. The processing device includes a processor or microprocessor capable of executing software. Typical examples can be computer processors (such as Intel® or AMD® processors), ASICs, microprocessors, and the like. The processor can be coupled to a memory, which can be typically a volatile RAM memory for storing instructions and data while the processor executes. The computer processor may also be coupled to a storage device, which can be a non-volatile storage medium, such as a hard drive, FLASH drive, tape drive, DVDROM, or similar device. Although not shown, the computer processing device typically includes various forms of input and output. The I/O may include network adapters, USB adapters, Bluetooth radios, mice, keyboards, touchpads, displays, touch screens, LEDs, vibration devices, speakers, microphones, sensors, or any other input or output device for use with a computer processing device. The computer processor may also be coupled to other type of computer-readable media, including, but are not limited to, an electronic, optical, magnetic, or other storage or transmission device capable of providing a processor, such as the processor, with computer-readable instructions. Various other forms of computer-readable media can transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript.

The program can be a computer program or computer readable code containing instructions and/or data, and can be stored on a storage device. The instructions may comprise code from any computer-programming language, including, for example, C, C++, C#, Visual Basic, Java, Python, Perl, and JavaScript. In a typical scenario, the processor may load some or all of the instructions and/or data of the program into the memory for execution. The program can be any computer program or process including, but not limited to a web browser, a browser application, an address registration process, an application, or any other computer application or process. The program may include various instructions and subroutines, which, when loaded into the memory and executed by the processor, cause the processor to perform various operations, some or all of which may effectuate the methods for managing medical care disclosed herein. The program may be stored on any type of non-transitory computer readable medium, such as, without limitation, hard drive, removable drive, CD, DVD or any other type of computer-readable media.

Certain aspects of the present disclosure include process steps and instructions described herein in the form of an algorithm. It should be noted that the process steps and instructions of the present disclosure could be embodied in software, firmware or hardware, and when embodied in software, could be downloaded to reside on and be operated from different platforms used by a variety of operating systems. The disclosure can also be in a computer program product which can be executed on a computing system.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a tangible computer-readable (or machine-readable) storage medium, such as, but not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. In some embodiments, the computer is connected to a display to display the images generated by the instant methods.

The algorithms and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description. In addition, the present disclosureis not described with reference to any particular programming language. It will be appreciated that a variety of programming languages may be used to implement the teachings of the present disclosure as described herein, and any references to specific languages are provided for disclosure of enablement and best mode of the present disclosure.

As will be understood by those familiar with the art, the invention may be embodied in other specific forms without departing from the spirit or essential characteristics thereof. Likewise, the particular naming and division of the modules, features, attributes, methodologies, managers and other aspects are not mandatory or significant, and the mechanisms that implement the invention or its features may have different names, divisions and/or formats. Furthermore, as will be apparent to one of ordinary skill in the relevant art, the modules, features, attributes, methodologies, managers and other aspects of the invention can be implemented as software, hardware, firmware or any combination of the three. Of course, wherever a component of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of skill in the art of computer programming. Additionally, the present invention is in no way limited to implementation in any specific programming language, or for any specific operating system or environment.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure.

1. A system for counting desired cells in a body fluid, comprising:
a. a microfluidic chip having a capture chamber for immobilizing the desired cells and receiving a labeling solution wherein the labeling solution interacts with an immobilized substance in the capture chamber and produces an optical signal;
b. an optoelectronic unit that detects the optical signal from the capture chamber that corresponds to the photochemical properties of the labeling molecule; and
c. a microprocessor that controls the microfluidic chip and the optoelectronic unit, and processes the optical signal measured by the optoelectronic unit to calculate a cell count.

2. The system according to claim 1, wherein the microfluidic chip comprises:
a. a first reservoir that collects the body fluid;
b. a section coated with an anti-coagulating agent;
c. a first mechanism that controls the volume of the body fluid;
d. the capture chamber;
e. a second mechanism that allows the body fluid to flow unidirectionally from the first mechanism to the capture chamber;
f. a first container of the labeling solution, which releases the labeling solution into the capture chamber; and
g. a second reservoir that collects waste.

3. The system according to claim 2, wherein the immobilized substance in the capture chamber comprises the desired cells, wherein the labeling solution interacts with the desired cells and produces the optical signal.

4. The system according to claim 2, further comprising a second container of a first wash solution, which releases the first wash solution into the capture chamber to wash away excess body fluid.

5. The system according to claim 2, wherein the anti-coagulating agent is Ethylenediaminetetraacetic acid (EDTA), citrate, heparin, vitamin E, or hementin.

6. The system according to claim 2, wherein the first mechanism comprises a reservoir with a gutter.

7. The system according to claim 2, wherein the second mechanism comprises a one-way flow valve or a programmable microfluidic pump.

8. The system according to claim 2, wherein the microfluidic chip further comprises a third container of an antibody-enzyme solution, which releases the antibody-enzyme solution into the capture chamber to react with the desired cells in the capture chamber.

9. The system according to claim 8, wherein the immobilized substance in the capture chamber comprises enzymes of the antibody-enzyme solution, and wherein the labeling solution reacts with the enzymes in the antibody-enzyme solution.

10. The system according to claim 8, wherein the microfluidic chip further comprises a fourth container of a second wash solution, which releases the second wash solution into the capture chamber to wash away excess antibody-enzyme solution.

11. The system according to claim 1, wherein the capture chamber comprises:
a. a solid scaffold; and
b. recognition molecules that selectively bind to the desired cells.

12. The system according to claim 11, wherein the substance in the capture chamber further comprises a linker, the linker comprising one chemical moiety or a linear arrangement of a plurality of chemical moieties, wherein the linker has strong binding affinity to the solid scaffold on one end and the recognition molecules on the other end.

13. The system according to claim 1, wherein the desired cells are cells with a unique surface molecular marker capable of being targeted.

14. The system according to claim 1, wherein the optoelectronic unit comprises:
a. a light source, the light source emitting light that that corresponds to the photochemical properties of the labeling molecule; and
b. an optical detection unit, the optical detection unit detecting the optical signal from the capture chamber that corresponds to the photochemical properties of the labeling molecule.

15. The system according to claim 2, further comprising a display and a memory that stores instructions, wherein the microprocessor processes the instructions to:
a. detect presence of the microfluidic chip;
b. detect entry of the body fluid into the capture chamber;
c. engage the first container after the desired cells are immobilized in the capture chamber to release the labeling solution;
d. receive the optical signal from the optical detection unit;
e. compare the optical signal to a pre-programmed algorithm to calculate the cell count; and
f. display the cell count on the display.

16. A method to count desired cells in a body fluid, comprising:
a. immobilizing the desired cells in a capture chamber on a microfluidic chip;
b. adding a labeling solution to the capture chamber, the labeling solution interacting with immobilized substance in the capture chamber and produces an optical signal;
c. detecting the optical signal from the capture chamber that correspond to the photochemical properties of the labeling molecule; and
d. providing a cell count based on the optical signal.

17. The method according to claim 16, wherein step (b) comprises
a. adding the labeling solution to the capture chamber; and
b. reacting the labeling solution with the immobilized substance in the capture chamber, wherein the immobilized substance in the capture chamber comprises desired cells, wherein the labeling solution interacts with the desired cells and produces the optical signal.

18. The method according to claim 16, wherein step (b) comprises
a. adding an antibody-enzyme solution to the capture chamber;
b. adding the labeling solution to the capture chamber; and
c. reacting the labeling solution with the immobilized substance in the capture chamber, wherein the immobilized substance in the capture chamber comprises enzymes in the antibody-enzyme solution.

19. The method according to claim 16, wherein step (c) comprises
a. emitting light that correspond to the photochemical properties of the labeling molecule; and
b. detecting the optical signal from the capture chamber constituting light that that correspond to the photochemical properties of the labeling molecule.

20. The method according to claim 16, wherein step (d) comprises
a. comparing the optical signal to a pre-determined algorithm to calculate the cell count; and
b. displaying the cell count.
